(19) United States
(12) Patent Application Publication
(10) Pub. No.: US 2022/0409113 A1
(43) Pub. Date: Dec. 29, 2022

(54) FEEDBACK LOOP FOR EMOTION RECOGNITION SYSTEM

(71) Applicant: Cephalgo SAS, Strasbourg (FR)

(72) Inventor: Hsin-Yin Chiang, Strasbourg (FR)

(21) Appl. No.: 17/847,137

(22) Filed: Jun. 22, 2022

Related U.S. Application Data
(60) Provisional application No. 63/214,285, filed on Jun. 24, 2021.

(51) Int. Cl. A61B 5/16; A61B 5/00; A61B 5/384; A61B 5/308

(52) U.S. Cl. CPC A61B 5/165; A61B 5/7264; A61B 5/384; A61B 5/308

(57) ABSTRACT

The present invention relates to a system and method of emotion recognition. An emotion recognition system may utilize a Valence-Arousal factor along with training data. The training data may exist as emotions assigned to actual measurements of user inputs. The actual measurements of user inputs may be assigned to a plurality of points on the Valence-Arousal model. A user input acquisition device may be used to collect actual measurements of user inputs. A processor may utilize an algorithm to assign user emotions based on the training data. A user may provide feedback on the assigned user emotions, and the training data may be updated based on the user feedback, depending on whether the user feedback is considered an outlier to the training data.

### BACKGROUND OF INVENTION

The present invention relates to the art of emotion recognition systems. In the art of emotion recognition systems, many systems exist that recognize the emotions of a user by use of various inputs. Common inputs are electroencephalographic (EEG) signals from a user's brain and electrocardiographic (ECG) signals from a user's heart. Many other inputs exist for said systems. Said other inputs include but are not limited to facial recognition and non-ECG heart rate input. Inputs are captured by various technologies, and are processed and analyzed by various technologies in order to recognize emotions.

Emotion recognition systems exist to provide objective recognition of emotions. Emotions of humans may be recognized by other humans without the use of technology by observing the facial expressions, movements, spoken words, and tones of voice of humans. These observations are then compared to the other humans' understanding of which facial expressions, movements, spoken words, and tones of voice correspond to certain emotions. The problem with these methods of recognizing emotions without the use of technology is that each human's understanding of humans' emotions is subjective and discontinuous. For several purposes, emotion recognition without the use of technology may lead to non-conclusive, non-objective outputs of emotion recognition.

For this reason, emotion recognition systems exist to obtain measurements of user inputs that are known in the art to change relative to a change in humans' emotions. For example, EEG signals from a user's brain are known to produce changing measurements as a user's emotions change. While a human may not be able to recognize slight changes in another human's emotions or in one's own emotions, measurements of EEG signals provide objective evidence of changing emotions. EEG signals can only be obtained from EEG devices that utilize EEG sensors, such as the EEG device described in U.S. patent application Ser. No. 17/227,355.

EEG devices, as well as other devices used to measure user inputs such as EGC devices and thermometers, provide the measurements of user inputs in the form of non-transient computer-readable media. While said non-transient computer-readable media can be configured into human-readable form for humans to process, it is much more effective to process non-transient computer-readable media using a processor. A processor is one or more electrical circuits with one or more switches that are able to process non-transient computer-readable media, since non-transient computer readable media exists as one or more electrical signals. For the purposes of this description, a “processor” shall mean the type of electric processor described herein, which is the type of processor used in computer-related systems and methods.

The emotion recognition systems that exist in the art utilize measurements of user inputs as well as a manual input from a user to recognize emotions. For example, an emotion recognition system may measure a user's brainwaves using EEG technology and ask a user to “manually” describe the current emotion that they feel. The user's manual answer is then equated with the EEG measurement. This form of emotion recognition is not without shortcomings. One major shortcoming is that different users may output different EEG signals for the same emotion. Besides physiological differences from user to user, this difference in EEG signal between users may be caused by the fact that emotions are measured qualitatively, whereas EEG signals and other inputs are measured quantitatively.

To overcome said shortcomings, artificial intelligence (AI) has been implemented in various systems to “learn” which qualitative emotions may be matched to certain quantitative input measurements. This may be achieved by measuring the inputs from multiple users and gaining manual feedback from multiple users. However, users may be poor at recognizing their own emotions, which is common among many humans, particularly patients with certain mental disorders. Such a system based only on user inputs and manual feedback can create ambiguity in the definition of emotions and therefore limits the types of emotions to be recognized and the accuracy with which they are recognized.

The main shortcoming of the AI emotion recognition systems previously described is that said systems do not account for the possible outliers in the manual feedback from users. In addition to limited emotions to be distinguished due to discrepancies in emotion definition, these systems may directly reply to the user's feedback, as the user's feedback can be biased due to possible incidents. Such incidents may be provision of incorrect entries, deliberately or not. Said incidents may lead to corruption of the existing database due to the input of inaccurate emotion recognition data. This shortcoming of not taking into account outliers in manual user feedback presents a major disadvantage when said AI emotion recognition systems are used to provide a service to users.

On the topic of providing a service to users; many of the emotion recognition systems in the art exist to provide a service to a user. Systems such as that described in KR101056793 provide tailored learning content to a user based on their current emotion. Other systems such as that described in KR20200057309 provide a report to a user that details the user's emotions throughout the day.

Combining the topics of outliers in manual user feedback and providing a service to users: The systems in the art previously described that use emotion recognition to provide a service to users may comprise manual user feedback in order to allow the emotion recognition systems to continuously learn how to interpret user inputs as a qualitative emotion. However, none of the systems that exist in the art take outliers into account when utilizing manual user feedback. This shortcoming could render the services provided by these systems biased or useless if incorrect manual user feedback is utilized by the system, since the system would gain an incorrect understanding of which user inputs measurements should be correlated with certain emotions.

Due to this and other shortcomings, there exists a need in the art for an AI emotion recognition system that takes into account manual user feedback, and further comprises the capability to distinguish outliers and/or inconsistencies within the manual user feedback, and the ability to discredit certain manual user feedback due to the system's understanding of emotion recognition.

### SUMMARY OF INVENTION

The present invention relates to an emotion recognition system that may utilize a Valence-Arousal model having a Valence factor with two endpoints and an Arousal factor with two endpoints. The Valence-Arousal model may further have a plurality of points, including the endpoints of the Valence factor and Arousal factor, and also including an origin, which is the intersection point between the Valence factor and the Arousal factor.

The Valence-Arousal model may be supplied with the user's feedback in form of self-assessment manikin within training data in the form of actual measurements of user inputs assigned to at least one of the plurality of points of the Valence-Arousal model. Said actual measurements of user inputs may include but are not limited to expected measurements of a user's heartrate, EEG waves, facial images, and/or body temperature. It is known in the art that EEG waves are electrical signals generated by a user's brain, and measurable via one or more EEG sensors of an EEG device. The training data may also comprise emotions assigned to each expected measurement of user inputs. For example, an emotion of relief may be assigned to an expected user measurement of a user's heartrate of 60 bpm, which may be assigned to one of the plurality of points of the Valence-Arousal model.

Training data may be obtained by collecting actual measurements of user inputs from a plurality of initial users. The number of initial users may be great enough to provide statistically valid training data.

The emotion recognition system may make use of one or more user acquisition devices that may be used to gather actual measurements of user inputs. Said user acquisition devices may include but are not limited to EEG devices, ECG devices, other heartrate monitors, and thermometers. The actual measurements of user inputs may be obtained from a live user via the one or more user acquisition devices.

The actual measurements of user inputs may be Hjorth parameters. Hjorth parameters are measurable factors such as activity, mobility, and complexity which are derived from EEG signals. Hjorth parameters give, among other things, statistical indications on the original EEG signal in the time domain.

The actual measurements of user inputs may be extracted in the frequency domain. The frequency domain representation of EEG signals is mainly obtained by calculating the Fourier transform of the EEG signals. From this representation in the Fourier domain, it is possible to calculate other types of quantities such as spectral entropy. This can be used to give an indication of the degree of disorder in the original signal. Other types of frequency domain representations such as wavelet decomposition or empirical mode decomposition can also be calculated from the actual measurements of user inputs.

The actual measurements of user inputs may be transmitted to a database in the form of non-transient computer-readable media. The actual measurements of user inputs may be retrieved from the database by a processor. The processor may use an algorithm to assign the actual measurements of user inputs to one or more of the plurality of points of the Valence-Arousal model. The processor may first classify the user inputs using the well-established objective standards of emotion derived by scientific and/or clinical studies and further use the algorithm to recognize the closest corresponding emotions to the one or more of the plurality of points to which the actual measurements of user inputs are assigned. The well-established objective standards of EEG brainwave interpretations, e.g. alpha implying a reflective or restful state, beta implying a busy active mind, and any advanced interpretations from further combinations of brainwaves, and common indicators of heartrate patterns for ECG, may serve as a general framework to firstly distinguish emotions, avoiding the scenarios that users have difficulties to correctly define emotions. The processor may further use the recognized closest corresponding emotions to assign user emotions, which are emotions that have been recognized by the emotion recognition system based on the actual measurements of user inputs and the training data.

The algorithm may be a convolution neural network The algorithm may further me a common convolution neural network known in the art such as a VGG16 convolution neural network. The algorithm may convert the actual measurements of user inputs into scalograms. A scalogram is the absolute value of the continuous wavelet transform (CWT) of an EEG signal, it is an image which is a time-frequency representation of the EEG signal. Therefore, it allows to keep the information of the original signal in the time and frequency domains at the same time.

The emotion recognition system may further comprise a user device to which the user emotions are sent in the form of non-transitory computer-readable media. The user emotions may be displayed on the user device in the form of human-readable text. Displaying the user emotions on the user device in the form of human-readable text may be in the form of a report to a user. In example embodiments, the report may detail which emotions the user has experienced during an 8, 12, or 24-hour time period. In addition to detailing which user emotions the user has experienced during the time period, the report may detail when the user has experienced each user emotion. For example, the report may detail the user emotions that the user experiences during an 8-hour time period from 12 pm to 8 pm. The report may state that the user experienced happiness from 12 pm-2 pm, boredom from 2 pm-5 pm, sadness from 5 pm-6 pm, and tiredness from 6 pm-8 pm.

The emotions mentioned herein such as happiness, boredom, etc. are used for purposes of example. Various embodiments of the invention are able to recognize various different emotions. It is understood that emotions may be defined differently across cultures, languages, etc. and thus a simple emotion label such as “happiness” may not be encompassing how the user actually feels. The AI technology and manual user feedback feature of the invention (described further herein) may enable the emotion recognition system to continuously “learn” various emotions and sub-emotions. For example, an emotion that the emotion recognition system may initially understand as happiness may be later understood by the invention to comprise sub-emotions such as content, pleasure, and overjoy. Said sub-emotions of happiness may be attributable to various measurements of user inputs within the range of measurements understood by the invention to correlate with happiness.

A user may use the user device to provide user's emotion feedback to the emotion recognition system. Providing user's emotion feedback may be referred to as “manual feedback” or “manual user feedback”. An example of manual user feedback is an example embodiment of the invention wherein an example emotion recognition system uses EEG measurements to recognize a user's emotions. The example emotion recognition system understands that sadness occurs with the designated characteristics of sadness in the user's brainwaves at 1 pm-2 pm. The user sees that the example emotion recognition system determined that the user experienced sadness from 1 pm-2 pm, when in reality the user was tired and not sad. The user may provide manual input to the processor in the form of non-transient, computer-readable media using the user device to tell the example emotion recognition system that from 1 pm-2 pm, the user felt tired instead of sad.

The processor may receive the user's emotion feedback (i.e. the manual user feedback) from the user device and may re-assign the emotions to re-assigned points on the Valence-Arousal model based on the user's emotion feedback. The processor may further update the algorithm based on the re-assigned points. In the previous example, the example emotion recognition system may determine that the user feels tiredness instead of sadness during future readings of actual measurements of user inputs if the example emotion recognition system receives EEG brainwaves that exhibit such characteristics.

The AI technology of the emotion recognition system would allow the system in the example to further define emotions in terms of measurements of user properties after being subjected to multiple rounds of user feedback. For example, the user in the previous example may provide multiple instances of manual user feedback throughout multiple uses of the system. These multiple instances of feedback may allow the example emotion recognition system to understand that tiredness occurs at one characteristic of brainwaves, and sadness occurs at another characteristic of brainwaves. Various embodiments may allow for the understanding of degrees of these emotions, such that the example emotion recognition system may understand through manual user feedback in more segmented emotions.

Manual user feedback may provide the shortcoming of causing the emotion recognition system to misunderstand emotions due to poor user feedback. Users of the present invention are intended to be human beings, and it is understood by those skilled in the art that many human beings possess poor emotion recognition skills compared to the general population. The users that will benefit from use of the present invention may be expected to have poor emotion recognition skills compared to the general population, which would provide a reason for them to use the present invention in order to understand their own emotions. For this reason, the present invention comprises segmented emotion recognition to guide the user to correctly provide the inputs, and an override feature that removes outliers in the manual user feedback.

The override feature may comprise a credibility algorithm that may determine that the user's emotion feedback provided by the user are outliers to the training data. The processor may not re-assign the emotions to re-assigned points on the Valence-Arousal model based on the user's emotion feedback if the user's emotion feedback are determined to be outliers by the credibility algorithm. Alternatively, the credibility algorithm may determine that the user's emotion feedback provided by the user are not outliers to the training data. In said cases, the processor may re-assign the emotions to re-assigned points on the Valence-Arousal model based on the user's emotion feedback.

To better describe the override feature, the previous example with the example emotion recognition system is reintroduced. In this example, the example emotion recognition system detects happiness, but the user provides a corrected user emotion of sadness. The example emotion recognition system may discredit this manual user feedback since it is grossly different from what the example emotion recognition system understands to be true. The action of discrediting a user's feedback may also involve the intervention or the verification by trained personnel or experts such as neurologists or psychiatrists.

The user input acquisition device may be an electroencephalographic (EEG) device. Said EEG device may be the EEG device of U.S. patent application Ser. No. 17/227,355, filed on Apr. 11, 2021, which is hereby incorporated by reference. The EEG device may comprise one or more flexible printed circuits on which are implemented one or more electroencephalographic sensors. The EEG device may utilize an electroencephalographic processing unit to process EEG signals acquired by the one or more electroencephalographic sensors. The one or more flexible printed circuits may be connected by a physical connection and/or by an electrical connection. A flexible material may be overmolded onto the one or more flexible printed circuits to serve as a barrier between the user and the one or more flexible printed circuits.

The EEG device may be worn around the user's ears and may be secured to the user's head by an adhesive. The adhesive may be pressure sensitive. The adhesive may further be a non-conductive biomimetic adhesive, which is defined in U.S. patent application Ser. No. 17/227,355.

In some embodiments, the number of the one or more electroencephalographic sensors may be at least 6, and at least some of said electroencephalographic sensors may contact the user's head at points FT9, FT10, T9, T10, A1, and A2 as they exist in a 10-10 electroencephalography system. In other embodiments, the number of the one or more electroencephalographic sensors may be at least 4, and at least some of said electroencephalographic sensors may contact the user's head at points FT9, FT10, T9, and T10 as they exist in a 10-10 electroencephalography system. In other embodiments, the number of the one or more electroencephalographic sensors may at least 4, and at least some of said electroencephalographic sensors may contact the user's head at points T9, T10, A1, and A2 as they exist in a 10-10 electroencephalography system.

The training data, algorithm, and credibility algorithm together may be referred to herein as “artificial intelligence”, “AI”, or “AI technology”. This is due to the fact that the combination of the training data, algorithm, and credibility algorithm allow the emotion recognition system to learn emotion recognition through feedback, said feedback being in the form of user's emotion feedback provided by a user.

The present invention is further related to a method of recognizing emotions. The method may utilize an emotion recognition system. Said emotion recognition system may be the emotion recognition system described herein. The method may comprise a Valence-Arousal model. The Valence-Arousal model may have a Valence factor and an Arousal factor, each with two endpoints. The Valence-Arousal model may further have a plurality of points including the endpoints of each Valence factor and Arousal factor. One of said points may be an origin, which is the point of intersection between the Valence factor and Arousal factor.

The method may further comprise assigning training data to the Valence-Arousal model. Said training data may exist as actual measurements of user inputs. The actual measurements of user inputs may be assigned to some of the plurality of points of the Valence-Arousal model, which may include the endpoints of each Valence factor and the Arousal factor. Assigning training data to the Valence-Arousal factor may further comprise assigning an emotion to each expected measurement of user inputs.

The method may further comprise providing a user input acquisition device and an algorithm. The user input acquisition device may be used for collecting actual measurements of user inputs. The method may further comprise transmitting the actual measurements of user inputs to a database in the form of non-transitory computer-readable media.

The actual measurements of user inputs may be denoised after being acquired by the user input acquisition device. Denoising may also be referred to as “pre-processing”. When the actual measurements of user inputs are EEG signals, said EEG signals may be acquired ast 128 Hz. EOG artifacts may then be removed before a bandpass frequency filter from 4-45 Hz is applied. The EEG data may then be averaged to a common reference. The denoised (clean) actual measurements of user inputs may be used to build input for a deep learning (AI) model without the need for additional data pre-processing.

The method may further comprise using a processor to retrieve the actual measurements of user inputs from the database. The method may further comprise using the processor to recognize one or more user emotions. Recognizing one or more user emotions may be achieved by using the algorithm to assign the actual measurements of user inputs to the one or more of the plurality of points of the Valence-Arousal model, using the algorithm to recognize the closest corresponding emotions to the one or more of the plurality of points, and assigning user emotions based on the closest corresponding emotions.

The method may further comprise transmitting the user emotions to a user device in the form of non-transitory computer-readable media. The method may further comprise displaying the user emotions on the user device in the form of human-readable text. The method may further comprise using the user device to provide user's emotion feedback. The method may further comprise re-assigning the emotions to re-assigned points on the Valence-Arousal model. The method may further comprise updating the algorithm based on the re-assigned points.

In some embodiments of the present invention, the method may comprise providing a credibility algorithm. The credibility algorithm may be used to determine that the user's emotion feedback are outliers to the training data. In these cases, the emotions may not be re-assigned to re-assigned points on the Valence-Arousal model, and the algorithm may not be updated. The credibility algorithm may alternatively be used to determine that the user's emotion feedback are not outliers to the training data. In these cases, the emotions may be re-assigned to re-assigned points on the Valence-Arousal model, and the algorithm may be updated.

In both the system and the method of the present invention, some of the re-assigned points may be the same as at least one of the some of the plurality of points to which actual measurements of user inputs are assigned. Alternatively, each of the re-assigned points may be a different point that any of the some of the plurality of points to which actual measurements of user inputs are assigned.

### BRIEF DESCRIPTION OF THE FIGURES

FIG. 1 is a first example Valence-Arousal model utilized by some embodiments of the present invention.

FIG. 2 is a second example Valence-Arousal model utilized by some embodiments of the present invention.

FIG. 3 is an example process flow of some embodiments of the present invention.

FIG. 4 is the example process flow of FIG. 3 wherein Emotion Recognition is further subdivided into 4 different processes.

FIG. 5 is an example logic flow utilized by some embodiments of the present invention for determining whether to accept or override manual user feedback.

FIG. 6 is an example process flow of a test period of an emotion recognition system.

FIG. 7 is an example process flow of creation of a user database.

FIG. 8 is an example process flow of denoising a user input such as an EEG signal.

FIG. 9 is an example process flow of validation of user credibility.

FIG. 10 is an example process flow of determination of a best AI model for emotion recognition.

FIG. 11 is an example Valence-Arousal model showing a distance between two emotions.

FIGS. 12-14 are an example process flow of a running period of an emotion recognition system.

FIG. 15 is an example process flow of emotion recognition by an AI model.

### DETAILED DESCRIPTION

The description provided herein describes example embodiments of the present invention and is not to be interpreted as limiting the invention to any particular embodiment, feature, step, or property. The figures provided and described herein also illustrate example embodiments of the invention, and are not to be interpreted as limiting the invention to any particular embodiment, feature, step, or property.

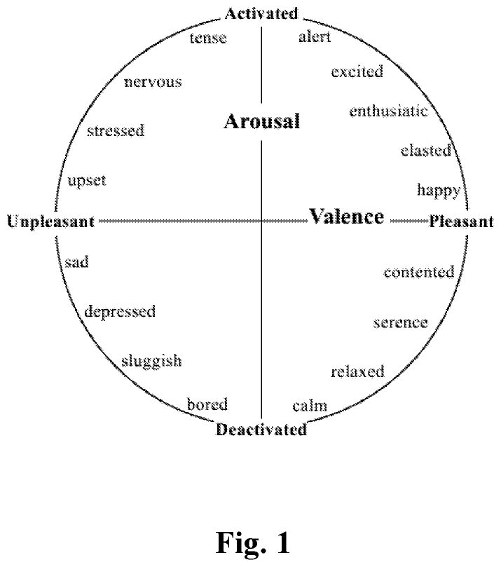

As shown in FIG. 1, a Valence-Arousal model has two axes: a Valence factor and an Arousal factor. Each axis has two endpoints. The endpoints of the Valence factor are labeled “Pleasant” and “Unpleasant”. The opposite ends of the Arousal factor are labeled “Activated” and “Deactivated”. The Valence-Arousal model allows emotions to be categorized into quadrants based on the Valence and Arousal axes. For example, in FIG. 1, the emotion “relaxed” falls in the quadrant defined by the Pleasant end of the Valence factor and the Deactivated end of the Arousal factor. This quadrant may be referred to as the “Pleasant-Deactivated” quadrant.

The Pleasant end of the Valence factor may be considered positive Valence, and the Unpleasant end of the Valence factor may be considered negative Valence. The Activated end of the Arousal factor may be considered positive Arousal, and the Deactivated end of the Arousal factor may be considered negative Arousal. The intersection of the Valence and Arousal axes may be referred to as the origin of the Valence-Arousal model, wherein the magnitude of both Valence and Arousal are zero. The halves of a Valence-Arousal model defined by each axis may be referred to as “hemispheres”. For example, in FIG. 1, tense and alert both fall into the Activated hemisphere, and happiness and content both fall into the Pleasant hemisphere. The two hemispheres created by the Valence factor may also be referred to as hemispheres.

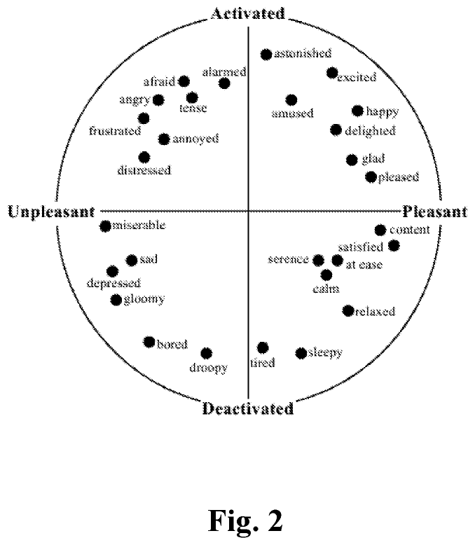

Due to the fact that Valence-Arousal models comprise finite ends to their axes, Valence-Arousal models may be depicted as circles, as shown in FIG. 1. FIG. 1 shows each emotion along the inner diameter of the circle. This is one example of a Valence-Arousal model. Another more detailed example of a Valence-Arousal model is shown in FIG. 2.

As shown in FIG. 2, different emotions are placed at different points within their respective quadrants in order to show the magnitude of both Valence and Arousal that each emotion comprises. Emotions located towards the origin of the graph may be considered to be more neutral emotions than the emotions located towards the edge of the graph. The Valence-Arousal model shown in FIG. 2 may be more informative and therefore better suited for use in certain emotion recognition applications.

The Valence-Arousal models illustrated in FIGS. 1 and 2 are examples of Valence-Arousal models and are not intended to limit the invention to utilizing any particular Valence-Arousal model or any particular emotion mapping. The locations of the emotions on the Valence-Arousal models utilized by the invention are subject to change based on the understanding at any given time of emotions in the art and how they should be graphed on a Valence-Arousal model. Furthermore, the present invention may modify a Valence-Arousal model utilized by the invention based on the invention's understanding at any given time of how emotions should be graphed on a Valence-Arousal model. The invention may perform said modifications as the invention's understanding of emotional graphing changes based on the “learning” of the AI feature of the invention.

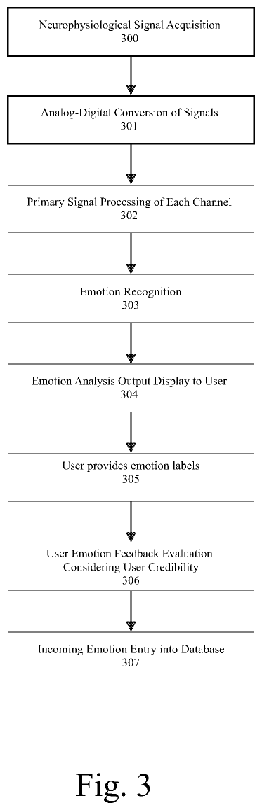

As shown in FIG. 3, a method of recognizing emotions begins with neurophysiological signal acquisition 300, which is using one or more user input acquisition devices to obtain actual measurements of user inputs. The actual measurements of user inputs are gathered as analog data and are then converted to digital data through analog-digital conversion of signals 301, the signals being the analog form of the actual measurements of user inputs. The method shown in FIG. 3 utilizes one or more EEG devices as the one or more user input acquisition devices. Since EEG devices provide multiple channels of actual measurements of user inputs, the method of recognizing emotions comprises primary signal processing of each channel 302, wherein each channel provided by the one or more EEG devices is processed separately by a processor.

Emotion recognition 303 is carried out by the processor utilizing the actual measurements of user inputs, a Valence-Arousal model, training data, and an algorithm. The output of emotion recognition 303 is user emotions that are provided to a user via emotion analysis output display to user 304. Emotion analysis output display to user 304 includes transmitting the user emotions to a user device in the form of non-transitory computer-readable media. The user may provide emotion labels 305 by using the user device to provide user's emotion feedback, which may be based on the user's own understanding of their own emotions. The method may further comprise utilizing a credibility algorithm for user emotion feedback evaluation considering user credibility 306, in which the credibility algorithm determines if the user's emotion feedback are considered outliers to the training data. Incoming emotion entry into database 307 includes entering either the user emotions or the user's emotion feedback into a database.

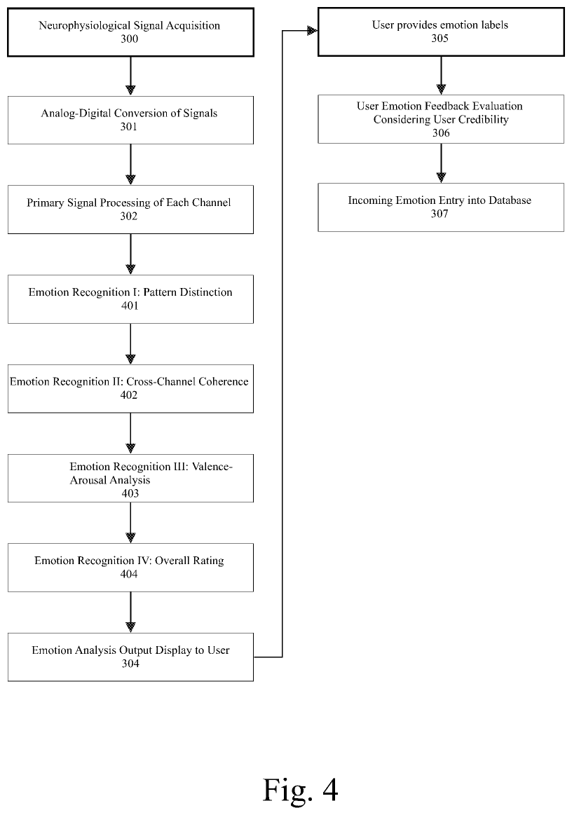

As shown in FIG. 4, emotion recognition 303 as used in the method of emotion recognition shown in FIG. 3 may be carried out by pattern distinction 401, cross-channel coherence 402, Valence-Arousal analysis 403, and overall rating 404. During pattern distinction 401, patterns within the actual measurements of user inputs obtained by the one or more EEG devices are recognized. During cross-channel coherence 402, the actual measurements of user inputs of each of the multiple channels provided by the one or more EEG devices are compared to one another to determine if the actual measurements of user inputs from each channel align with one another.

During Valence-Arousal analysis 403, a processor uses an algorithm to assign the actual measurements of user inputs to one or more of a plurality of points of a Valence-Arousal model. The processor then uses the algorithm to recognize the closest corresponding emotions, said emotions having been assigned to some of the plurality of points as part of the training data. The processor then assigns user emotions based on the closest corresponding emotions during overall rating 404.

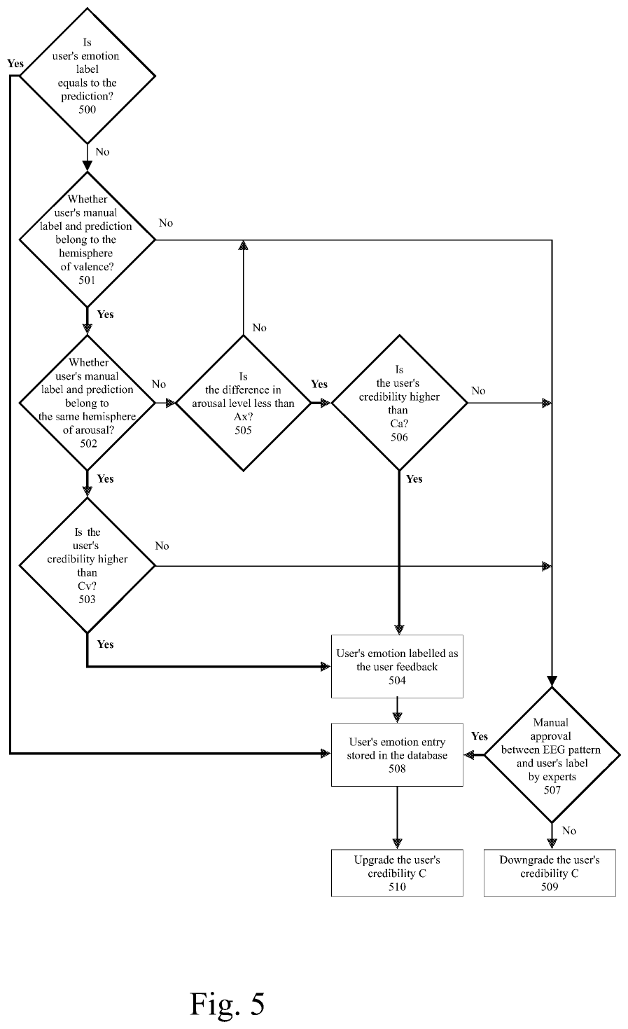

As shown in FIG. 5, a logic flow may be utilized by a credibility algorithm in order to determine if the user's emotion feedback provided by the user are outliers to the training data. First, it is determined whether the user's emotion feedback (user's emotion label) is an exact match to the user emotions recognized (equals to the prediction) 500. If so, the user's emotion feedback is stored in a database 508 and the user's credibility “C”, is increased 510.

If the user's emotion feedback are not an exact match to the user emotions recognized 500, it is determined whether the user's emotion feedback and the user emotions provided belong to the same hemisphere of Valence 501. If not, the user's emotion feedback are subject to manual review by experts 507, said experts being humans trained in the art of emotion recognition such as physiatrists. If the manual review by experts 507 determines that the user's emotion feedback are valid, then the user's emotion feedback are stored in the database 508 and the user's credibility is increased 510. If the manual review by experts 507 determines that the user's emotion feedback are invalid, then the user's emotion feedback are not entered into the database and the user's credibility is decreased 509.

If the user's emotion feedback and the user emotions provided belong to the same hemisphere of Valence 501, it is then determined if the user's emotion feedback and user emotions provided belong to the same hemisphere of Arousal 502. If so, it is then determined if the user's credibility “C” is higher than Cv 503. If C is higher than Cv, the emotions of the training data are re-assigned to re-assigned points on the Valence-Arousal model and the algorithm is updated based on the re-assigned points 504. The user's emotion feedback is entered into the database 508 and C is increased 510.

If, when determining if C is higher than Cv 503, it is determined that C is lower than Cv, the user's emotion feedback are subject to manual review by experts 507. If the manual review by experts 507 determines that the user's emotion feedback is valid, then the user's emotion feedback are stored in the database 508 and C is increased 510. If the manual review by experts 507 determines that the user's emotion feedback are invalid, then the user's emotion feedback are not entered into the database and the user's credibility is decreased 509.

If the manual user feedback and the emotion recognition of the invention do not belong to the same hemisphere of Valence 501 nor the same hemisphere of Arousal 502, the difference in Arousal level between the user's emotion feedback and the user emotions provided is calculated and compared to Ax 505. If said difference is greater than Ax, the user's emotion feedback is subject to manual review by experts 507. If the manual review by experts 507 determines that the user's emotion feedback is valid, then the user's emotion feedback is stored in the database 508 and C is increased 510. If the manual review by experts 507 determines that the user's emotion feedback are invalid, then the user's emotion feedback are not entered into the database and the user's credibility is decreased 509.

If the difference in Arousal level between the user's emotion feedback and the user emotions provided 505 is less than Ax, C is compared to Ca 506. If C is greater than Ca, the emotions of the training data are re-assigned to re-assigned points on the Valence-Arousal model and the algorithm is updated based on the re-assigned points 504. The user's emotion feedback are entered into the database 508 and C is increased 510. If C is less than Ca, the user's emotion feedback is subject to manual review by experts 507. If the manual review by experts 507 determines that the user's emotion feedback is valid, then the user's emotion feedback is stored in the database 508 and C is increased 510. If the manual review by experts 507 determines that the user's emotion feedback are invalid, then the user's emotion feedback are not entered into the database and the user's credibility is decreased 509.

Cv, C, Ca, and Ax are used in this description and are known statistical properties in the art of statistics. Thus, the terms Cv, C, Ca, and Ax shall be interpreted by their meaning in the art of statistics for purposes of this description.

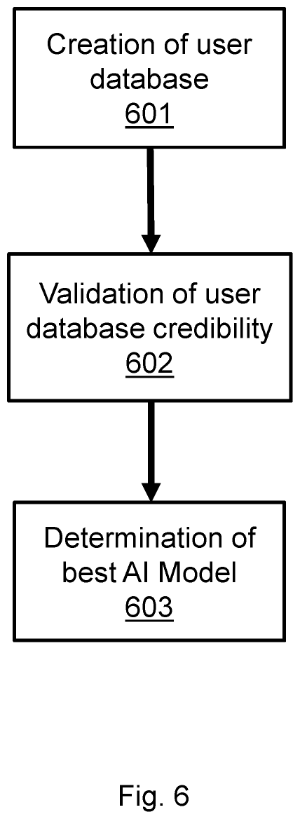

The emotion recognition system of the present invention may comprise a test period (also referred to as a “reference period”) and a running period. As shown in FIG. 6, the test period may start with the creation of a user database 601. The user database may be tested (“validation of user database credibility”) 602, and an appropriate AI model for emotion recognition may be determined (“determination of best AI model”) 603. Validation of user database credibility 602 may consist of parts such as but not limited to checking the consistency of user's emotion feedback upon the same emotion or upon the signal characteristics and verifying the user's emotion feedback using the objective standards of emotion derived by scientific and/or clinical studies. The test period may be repeated any number of times in order to provide the emotion recognition system with enough training data to use during the running period. During the test period, a database may be created for each user of the emotion recognition system thereby creating a “subject dependent” emotion recognition system. Alternatively, a single database may be created for a plurality of users, thereby creating a “subject independent” emotion recognition system.

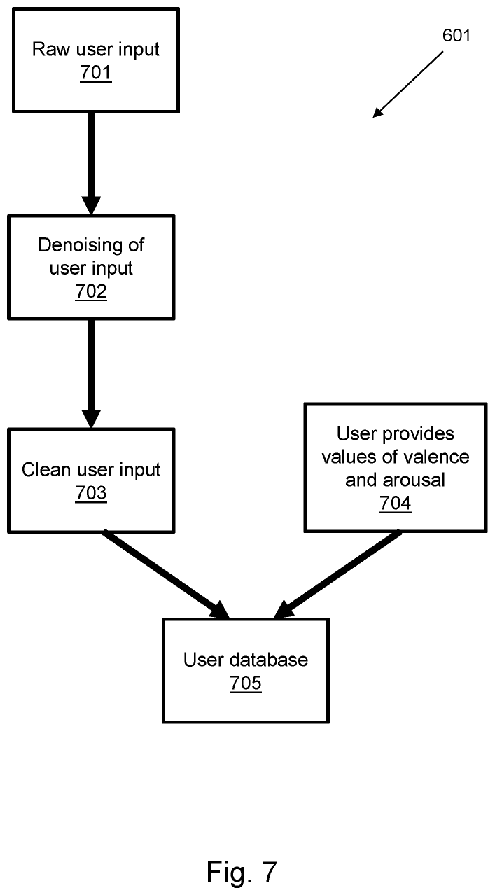

As shown in FIG. 7, creation of the user database 601 may start with raw user input 701. Raw user input 701 may exist as un-filtered or otherwise un-altered user input such as but not limited to EEG signals, fNIRS signals, ECG signals, and body temperature. The raw user input 701 is denoised through denoising of user input 702, which produces clean user input 703. A user may provide initial values of Valence and Arousal 704 (also referred to as “initial user annotations”) based on the values of Valence and Arousal that the user thinks correlate with their emotions. The clean user input 703 and initial user annotations 704 are then added to the user database 705.

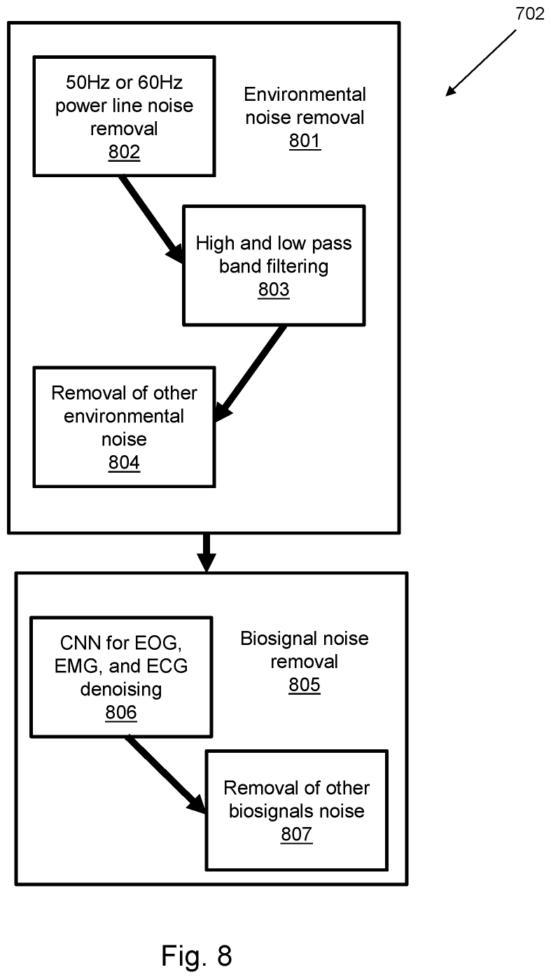

As shown in FIG. 8, denoising of user input 702 may comprise environmental noise removal 801 and biosignals noise removal 805. FIG. 8 shows biosignals noise removal 805 being completed after environmental noise removal 801. However, various embodiments of the invention may utilize various denoising techniques. Some embodiments of the invention may only utilize environmental noise removal 801 or biosignals noise removal 805. Other embodiments of the invention may complete biosignals noise removal 805 before environmental noise removal 801.

During environmental noise removal 801, 50 Hz or 60 Hz power line noise removal 802 may occur, during which portions of the raw user input that have frequencies of exactly 50 Hz or 60 Hz are removed from the user input. These specific frequency values are chosen since noise created by EEG devices is generally at the frequency of 50 Hz in European EEG devices and 60 Hz in American EEG devices. High pass and low pass band filtering 803 may also occur as part of environmental noise removal 801. During high pass and low bass band filtering 803, portions of the raw user input in the range of 0-4 Hz inclusive (low pass band) and portions of the raw user input in the range of 45-128 Hz inclusive (high pass band) are removed from the user input. These frequency ranges are chosen since they correlate with common environmental noise generated by events such as but not limited to the user moving or the user touching the user input acquisition device. Removal of other environmental noise 804 may further occur as part of environmental noise removal 801.

During biosignals noise removal 805, unwanted user input may be removed from the raw user input by CNN for EOG, EMG, and ECG denoising 806. In emotion recognition systems that utilize EEG as the preferred user input, other user inputs such as eye blink artifacts (EOG), muscle artifacts (EMG), and heartbeat artifacts (ECG) may be considered noise, and therefore should be removed from the user input. This may be achieved using a convolutional neural network (CNN) which is trained to recognize EEG signals when mixed with other biosignals such as EOG, EMG, and ECG. A convolutional neural network is a series of AI algorithms that are configured to recognize 1-dimensional (1D) images. The “algorithm” referred to herein may be a convolutional neural network. The “credibility algorithm” referred to herein may also be a convolutional neural network. Examples of CNNs that exist in the art are GoogLeNet, VGG16, and VGG19. During CNN for EOG, EMG, and ECG denoising 806, the various biosignals such as EEG, EOG, EMG, and ECG may be converted into 2D images that may be recognized using the CNN. The CNN may then separate the EEG signal from the rest of the biosignals, thereby removing the rest of the biosignals from the user input.

Removal of other biosignals noise 807 may also occur as part of biosignals noise removal 805.

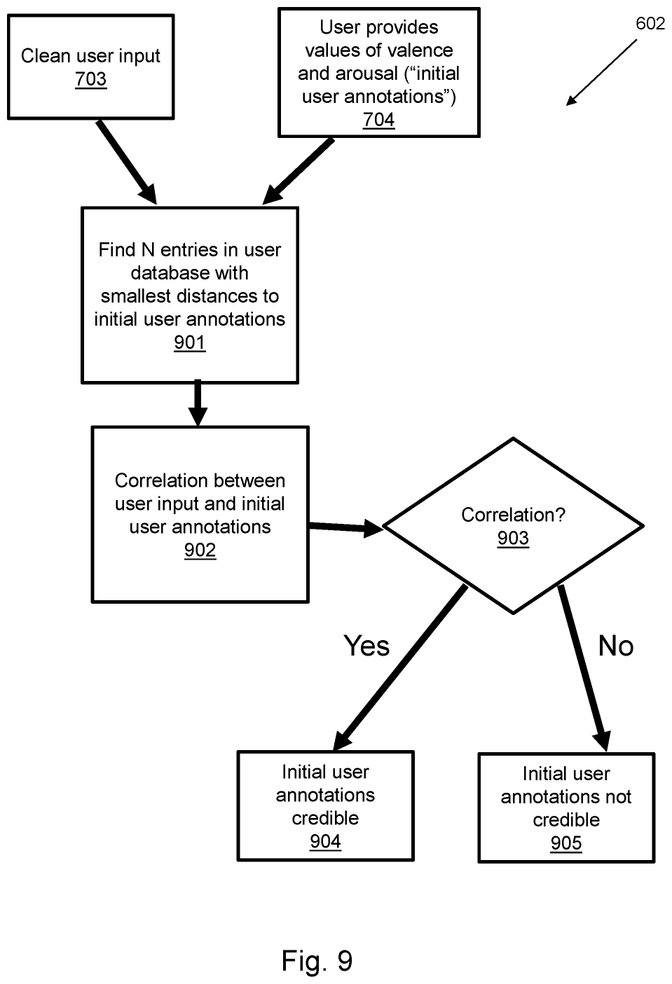

As shown in FIG. 9, a user provides values of Valence and Arousal (initial user annotations) 704, which are compared to the clean user input 703, as well as existing user input in the user database. The emotion recognition system finds a number (N) entries within the user database with the smallest distances to the initial user annotations 901. An algorithm, which may be a CNN, determines the correlation between the user input and the initial user annotations 902. If a high correlation is determined by the algorithm, the initial user annotations are said to be credible 904. If a negative correlation is determined by the algorithm, the initial user annotations are said to be not credible 905.

To determine the correlation between the user input and initial user annotations 902, the algorithm may calculate a correlation coefficient between the clean user input 703 and initial user annotations, as well as correlation coefficients between the initial user annotations and other user input entries that exist in the user database. Of the correlation coefficients between the initial user annotations and the other user input entries, an average correlation coefficient is calculated. A correlation criterion is then used to determine if the average correlation coefficient between the initial user annotations and existing user input entries is correlated to the correlation coefficient of the initial user annotations and the new, clean user input 703.

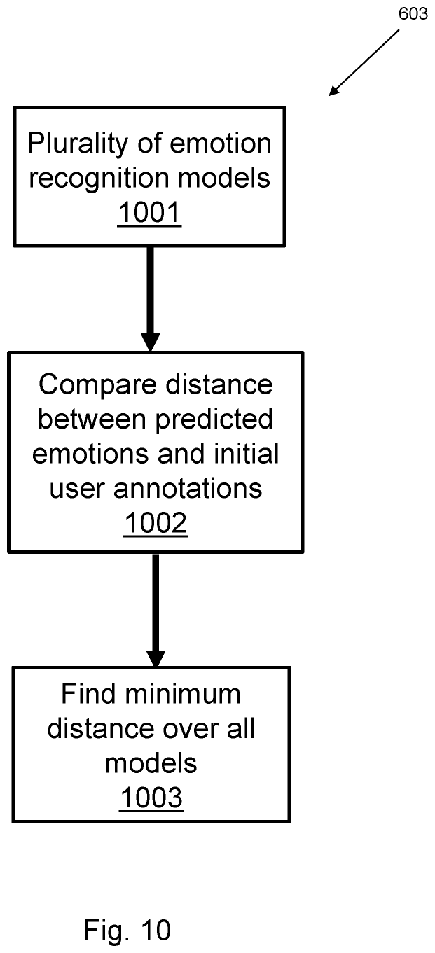

As shown in FIG. 10, determination of best AI model 603 may use a plurality of emotion recognition models 1001. The algorithm compares the distances between the predicted emotions of each of the plurality of emotion recognition models to the initial user annotations 1002. The minimum distance is then found over all of the emotion recognition models 1003, and the emotion recognition model with the smallest distance is chosen as the best AI model.

Determination of best AI model 603 is particularly useful for subject dependent emotion recognition systems, since different AI models may work better for different users. Therefore, the same emotion recognition system may be used by multiple different users even though different AI models are used for each user.

The “AI model” described herein may comprise the algorithm and credibility algorithm described herein. Either, or both, of the algorithm and credibility algorithm may be a convolutional neural network.

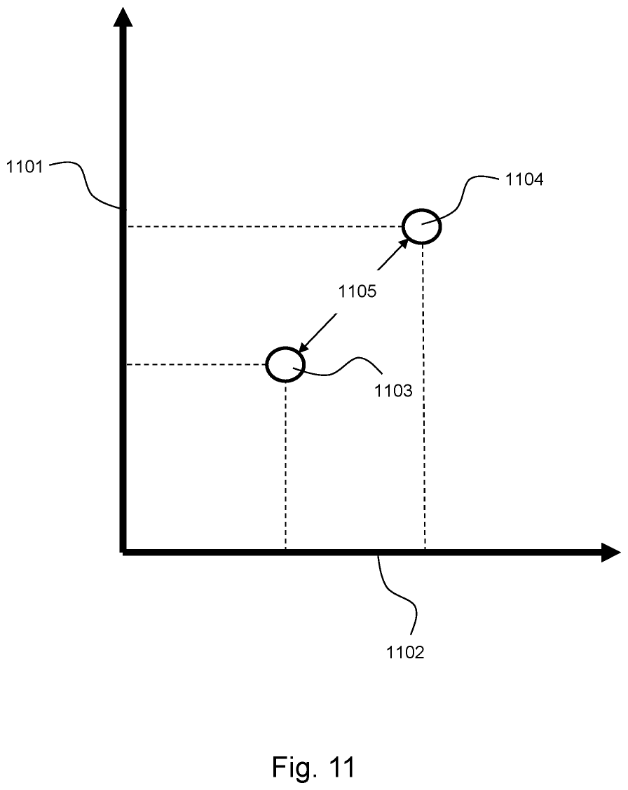

As shown in FIG. 11, a 2D Valence-Arousal model is used with an Arousal factor 1101 and a Valence factor 1102. A first emotion 1103 and second emotion 1104 are assigned two different points within the Valence-Arousal model. The distance 1105 between the first and second emotions is calculated using the Euclidian distance formula:

d=\sqrt{α(V2−V1)²+β(A2−A1)²}

wherein d is the distance between the first and second emotions, V1 is the Valence value of the first emotion, V2 is the Valence value of the second emotion, A1 is the Arousal value of the first emotion, A2 is the Arousal value of the second emotion, α is a Valence constant, and β is an Arousal constant. The Valence and Arousal constants may be used to weigh either Valence or Arousal. For example, if Valence is determined to be twice as important for emotion recognition, the Valence constant may be set to twice the value of the Arousal constant.

The first and second emotions may be two emotions of the same user in the database. For example, a User A may provide two readings of a happiness emotion to the database, which may be the first and second emotions. The first and second emotions may alternatively be two emotions from different users in the database. For example, a User A may provide a reading of happiness and a User B may also provide a reading of happiness, which may be the first and second emotions. The first and second emotions may alternatively be an initial user annotation and a user emotion. For example, a User A may provide a reading of happiness (the first emotion), and may also provide an annotation of User A's Valence and Arousal values (the second emotion).

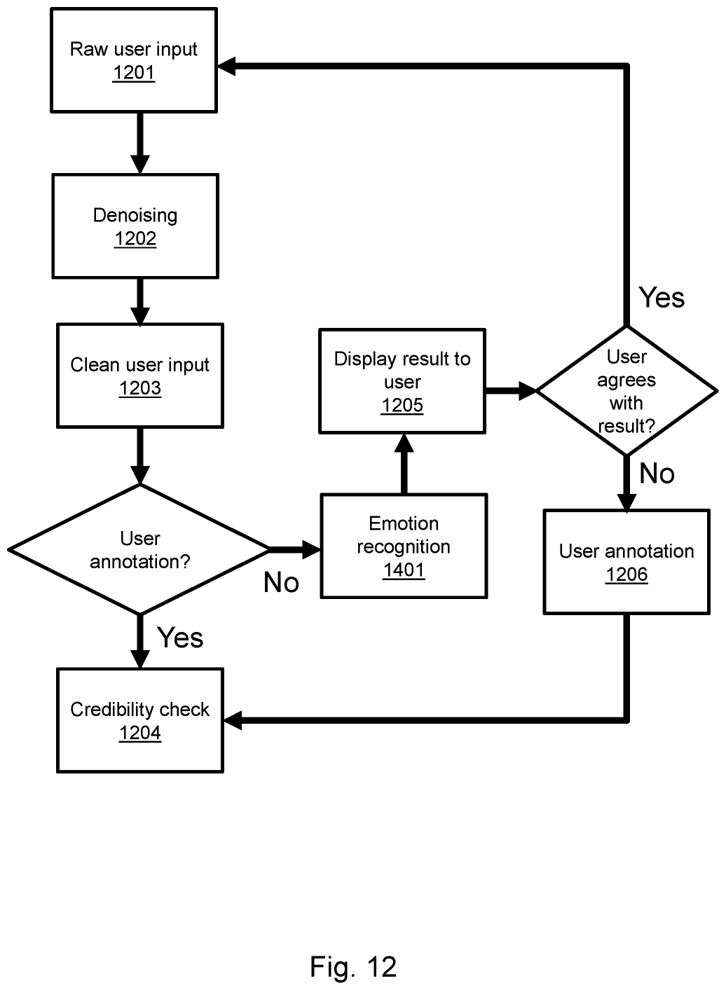

As shown in FIG. 12, the running period may start with raw user input 1201. Denoising 1202 may occur, which may function exactly like the denoising of the test period, resulting in a clean user input 1203. If user annotation 1206 has been provided, a credibility check 1204 occurs. The credibility check 1204 may function exactly like the credibility check (correlation determination) of the test period. If no user annotation 1206 has been provided, the running period proceeds to emotion recognition 1401. The results of emotion recognition 1401 are displayed to the user 1205. If the user agrees with the results of emotion recognition 1401, the running period continues as a new cycle with new raw user input 1201. If the user does not agree with the results of emotion recognition 1401, user annotation 1206 is provided, and a credibility check 1204 is performed on the user annotation 1206. User annotation 1206 may be values of Valence and Arousal provided by a user, said values being the values of Valence and Arousal that the user thinks correspond with their emotions.

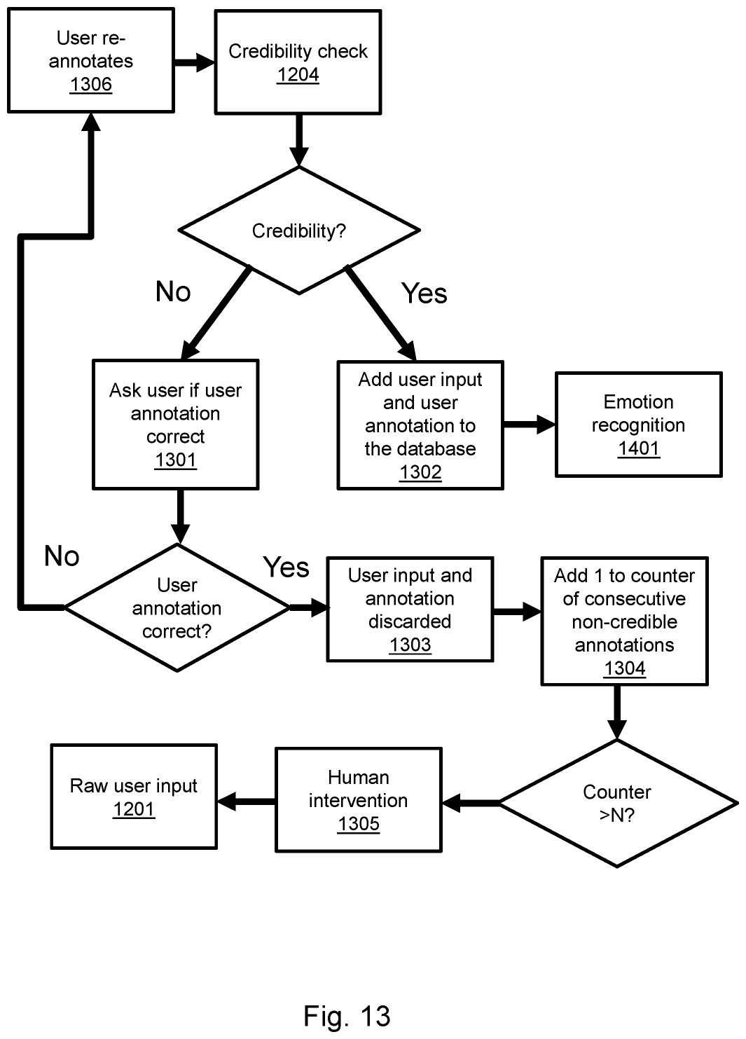

As shown in FIG. 13, if there is no correlation/credibility between the clean user input 1203 and the user annotation 1206, then the user is asked if the user annotation is correct 1301. Asking the user to re-annotate 1301 may prevent mistakes in user annotation from contaminating the database with incorrect data. If the user states that their previous annotation is not correct, the user re-annotates 1306, and their re-annotation is subjected to a credibility check 1204. If the user states that their previous annotation is correct, the user input and user annotation are discarded 1303. 1 count is then added to a counter of consecutive non-credible annotations 1304. If the total count is found to be greater than a number N, then human intervention 1305 is required, and the running period starts over from obtaining raw user input 1201.

The number N may be any positive integer. The number N may be chosen to represent a number of consecutive non-credible user annotations that would determine a user to be incapable of providing accurate user annotations, therefore requiring human intervention 1305. Human intervention 1305 may be performed by medical experts in the art such as psychologists or psychiatrists.

Also as shown in FIG. 13, if there is correlation/credibility between the user input and user annotation, the user input and user annotation are entered into the database 1302, and the running period proceeds to emotion recognition 1401.

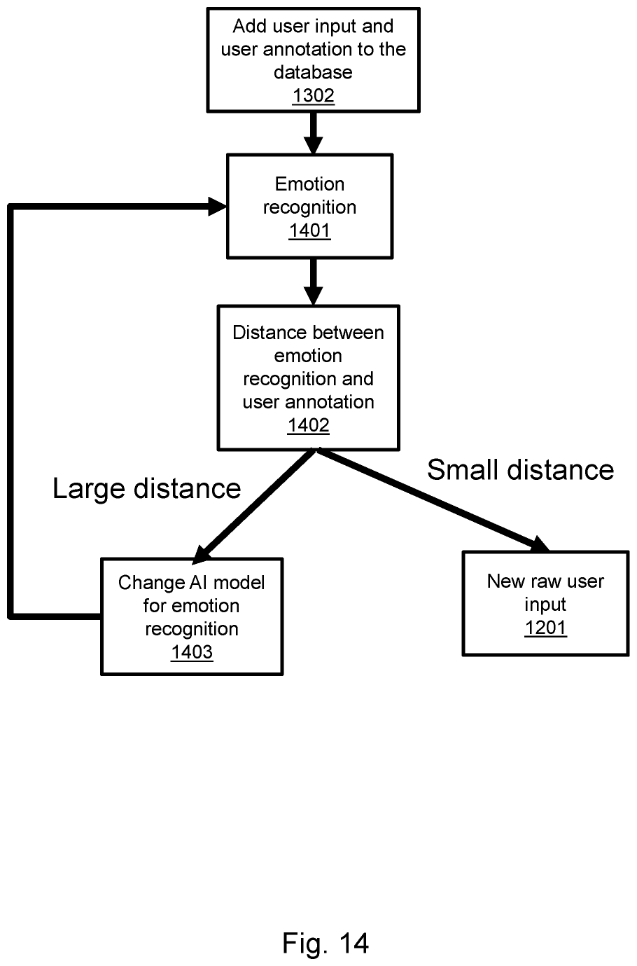

As shown in FIG. 14, the emotion recognized by the emotion recognition system is compared to the user annotation 1402. This is done in order to determine the best AI model for emotion recognition. Though an AI model was previously used during emotion recognition 1401, calculating the distance between the recognized emotion and the user annotation 1402 may determine whether said AI model is the best AI model to be used for a particular user. A set criterion may be used to determine if the distance between the recognized emotion and the user annotation 1402 is “large” or “small”. If the distance is small, the recognized emotion is considered valid, and the emotion recognition system may recognize further emotions by obtaining new raw user input 1201. If the distance is large, the AI model for emotion recognition is changed 1403 based on the AI model that provides the smallest distance between the recognized emotion and the user annotation. Upon changing the AI model for emotion recognition 1403, the running period resumes from emotion recognition 1401.

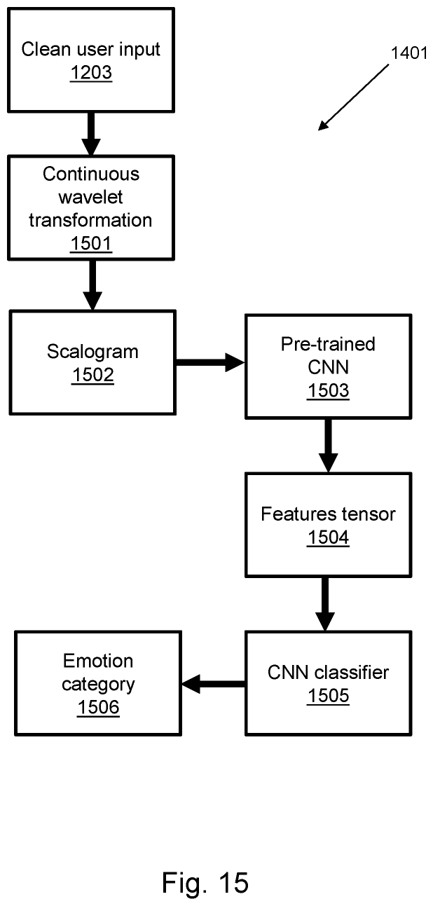

As shown in FIG. 15, emotion recognition 1401 may be completed using clean user input 1203. Since emotion recognition uses an algorithm that may be a convolution neural network, continuous wavelet transformation 1501 is used to convert the 1-dimensional (1D) clean user input 1203 into a 2D scalogram 1502 that can be read by a CNN. A pre-trained CNN 1503 (CNN that has been equipped with training data such as that acquired during the test period of the emotion recognition system). The scalogram 1502 may be a time-frequency representation of the clean user input 1203. Within the pre-trained CNN 1503, a tensor of a predetermined size encapsulates the relevant feature of the scalogram necessary for recognizing an emotion. Said tensor may be the input to a second CNN (CNN classifier 1505) that is dedicated to using the image provided by said tensor to output an emotion category 1506, thereby recognizing an emotion using clean user input 1203. The CNN classifier 1505 may be part of the overall pre-trained CNN 1503.

What is claimed is:

1. An emotion recognition system comprising:
a. a Valence-Arousal model comprising:
i. a Valence factor comprising two endpoints;
ii. an Arousal factor comprising two endpoints;
iii. a plurality of points, one of said plurality of points being an origin;
b. an algorithm;
c. a user input acquisition device;
d. a database comprising training data, said training data comprising:
i. actual measurements of user inputs assigned to the endpoints of each the Valence factor and the Arousal factor;
ii. actual measurements of user inputs assigned to some of the plurality of points, said some of the plurality of points being in addition to the endpoints of the Valence factor and the Arousal factor;
iii. emotions assigned to each actual measurement of user inputs;
e. a processor; and
f. a user device,
wherein the user input acquisition device collects actual measurements of user inputs, and wherein the actual measurements of user inputs are transmitted to the database in the form of non-transitory computer-readable media,
and wherein the processor retrieves the actual measurements of user inputs from the database,
and wherein the processor uses the algorithm to assign the actual measurements of user inputs to one or more of the plurality of points of the Valence-Arousal model, and wherein the processor uses the algorithm to recognize the closest corresponding emotions to the one or more of the plurality of points,
and wherein the processor uses the algorithm to assign user emotions based on the closest corresponding emotions to said one or more of the plurality of points, and wherein the user emotions are transmitted to a user device in the form of non-transitory computer-readable media,
and wherein the user emotions are displayed on the user device in the form of human-readable information,
and wherein a user uses the user device to provide user's emotion feedback.

2. The emotion recognition system of claim 1, further comprising a credibility algorithm, wherein the credibility algorithm determines that the user's emotion feedback are outliers, and wherein the user's emotion feedback are discarded.

3. The emotion recognition system of claim 1, further comprising a credibility algorithm, wherein the credibility algorithm determines that the user's emotion feedback are not outliers,
and wherein the processor re-assigns the emotions to re-assigned points on the Valence-Arousal model based on the user's emotion feedback,
and wherein the processor updates the algorithm based on the re-assigned points.

4. The emotion recognition system of claim 1, wherein the user input acquisition device is an EEG device.

5. The emotion recognition system of claim 1, wherein the user input acquisition device in an ECG device.

6. The emotion recognition system of claim 1, wherein the actual measurements of user inputs are transformed into Hjorth parameters for further processing.

7. The emotion recognition system of claim 1, wherein the Fourier transform is applied to the actual measurements of user inputs to obtain other measurements of user inputs.

8. The emotion recognition system of claim 1, wherein the processor uses the algorithm to assign the actual measurements of user inputs to one or more of the plurality of points of the Valence-Arousal model by converting the actual measurements of user inputs into one or more scalograms or other continuous wavelet transformation coefficient(s) as the input of machine learning/deep learning.

9. The emotion recognition system of claim 8, wherein one or more pre-trained algorithms such as VGG16 is used within the convolution neural network.

10. A method of recognizing emotions comprising:
a. Providing a Valence-Arousal model, the Valence-Arousal model comprising:
i. a Valence factor comprising two endpoints;
ii. an Arousal factor comprising two endpoints;
iii. a plurality of points, one of said plurality of points being an origin;
b. assigning training data to the Valence-Arousal model, comprising:
i. assigning actual measurements of user inputs to the endpoints of each the Valence factor and the Arousal factor;
ii. assigning actual measurements of user inputs to some of the plurality of points, said some of the plurality of points being in addition to the endpoints of the Valence factor and the Arousal factor;
iii. assigning an emotion to each expected measurement of user inputs;
c. providing an algorithm;
d. providing a user input acquisition device;
e. collecting actual measurements of user inputs with the user input acquisition device;
f. providing a processor;
g. using the processor to denoise the actual measurements of user inputs;
h. transmitting the actual measurements of user inputs to a database in the form of non-transitory computer-readable media;
i. using the processor to retrieve the actual measurements of user inputs from the database;
j. using the processor to recognize one or more user emotions by:
i. using the algorithm to assign the actual measurements of user inputs to one or more of the plurality of points of the Valence-Arousal model;
ii. using the algorithm to recognize the closest corresponding emotions to the one or more of the plurality of points;
iii. assigning user emotions based on the closest corresponding emotions;
k. transmitting the user emotions to a user device in the form of non-transitory computer-readable media;
l. displaying the user emotions on the user device in the form of human-readable text; and
m. using the user device to provide user's emotion feedback.

11. The method of claim 10, further comprising providing a credibility algorithm, wherein after using the user device to provide user's emotion feedback, the credibility algorithm is used to determine that the user's emotion feedback are outliers, and wherein the user's emotion feedback are discarded.

12. The method of claim 10, further comprising providing a credibility algorithm, wherein after using the user device to provide user's emotion feedback, the credibility algorithm is used to determine that the user's emotion feedback are not outliers, and wherein the emotions are re-assigned to re-assigned points on the Valence-Arousal model, and wherein the algorithm is updated based on the re-assigned points.

13. The method of claim 12, wherein said method is continuously repeated.

14. The method of claim 10, wherein the user inputs device is an EEG device.

15. The method of claim 10, wherein the user inputs device is an ECG device.

16. The emotion recognition system of claim 10, wherein the actual measurements of user inputs are transformed into Hjorth parameters for further processing.

17. The emotion recognition system of claim 10, wherein the Fourier transform is applied to the actual measurements of user inputs to obtain other measurements of user inputs.

18. The emotion recognition system of claim 17, further comprising denoising the other measurements of user inputs.

19. The emotion recognition system of claim 1, wherein the processor uses the algorithm to assign the actual measurements of user inputs to one or more of the plurality of points of the Valence-Arousal model by converting the actual measurements of user inputs into one or more scalograms.

20. The emotion recognition system of claim 10, wherein one or more pre-trained algorithms such as VGG16 is used within the convolution neural network.
